(19) United States
(12) Patent Application Publication
(10) Pub. No.: US 2025/0147019 A1
(43) Pub. Date: May 8, 2025

(54) BIOTIN-TRAP BEADS AND METHODS OF PRODUCTION AND USE THEREOF

(71) Applicant: Siemens Healthcare Diagnostics Inc., Tarrytown, NY (US)

(72) Inventors: Zhu Teng, Garnet Valley, PA (US); Jesse Spillane, Newark, DE (US); Jie Li, Middletown, DE (US); Thomas Kennedy, Newark, DE (US); Karen Krakowski, Chesapeake City, MD (US); Izak Bahar, Hockessin, DE (US); Tie Wei, Wilmington, DE (US)

(73) Assignee: Siemens Healthcare Diagnostics Inc., Tarrytown, NY (US)

(21) Appl. No.: 18/715,400

(22) PCT Filed: Dec. 16, 2022
(86) PCT No.: PCT/US2022/081793

§ 371 Date: May 31, 2024

Related U.S. Application Data
(60) Provisional application No. 63/265,621, filed on Dec. 17, 2021.

(51) Int. Cl. G01N 33/543; G01N 33/533; G01N 33/548

(52) U.S. Cl. CPC G01N 33/54386; G01N 33/533; G01N 33/54353; G01N 33/548

(57) ABSTRACT

Biotin-trap compositions are disclosed that contain particles having an inner polymer coating layer disposed on at least a portion of an outer surface thereof and a biotin-specific binding partner conjugated to the inner polymer coating layer. The biotin-trap compositions may further include an outer polymer coating layer disposed about the biotin-specific binding partner. The biotin-trap compositions specifically bind to free biotin but do not substantially bind to biotin conjugated to other moieties, such as biotinylated assay reagents. Also disclosed are kits and microfluidics devices that include the biotin-trap compositions, as well as methods of producing and using the biotin-trap compositions.

### CROSS REFERENCE TO RELATED APPLICATIONS/INCORPORATION BY REFERENCE STATEMENT

This application claims priority of U.S. Provisional application No. 63/265,621 filed 17 Dec. 2021, the contents of which are fully incorporated herein by reference.

### FIELD

Embodiments of the present disclosure generally relate to compositions, kits, and methods for the removal of specifically-bindable substances from a biological sample.

### BACKGROUND

Diagnostic assay reagents often include conjugates of antibodies or other small drug molecules with haptens, such as biotin and fluorescein. The tight binding of these haptens to large protein molecules (e.g., avidin/streptavidin for biotin and anti-FITC for fluorescein) that are coated on a solid support or surface provides a convenient way to immobilize the hapten-antibody or hapten-drug conjugate on the solid support/surface.

Biotin is known in the art for its use as a food supplement; for example, biotin is utilized to promote healthy hair and nail growth and to treat various disease conditions. Given this use, significant biotin levels can be found in biological samples, such as (but not limited to) blood. Since biotin is used in many diagnostic assays (for example, to coat solid supports), high levels of biotin in test samples can interfere with assay signals in any assays where biotinylated assay components are employed. This is particularly true for assays such as those found on automated diagnostic test instruments such as the Siemens' ADVIA CENTAUR® immunoassay system, DIMENSION EXL™ integrated chemistry system, and DIMENSION VISTA® LOCI® system (Siemens Healthcare Diagnostics Inc., Tarrytown, NY), where biotinylated assay components are expected to bind streptavidin-coated solid supports.

One method for handling interference in various assay methods that utilize biotin as part of an assay reagent is to quantitate the level of biotin present in patient samples to determine if the samples are suitable for use in particular assays. However, most currently available biotin assays have narrow ranges (i.e., 0-50 ng/ml) that are not suitable for detecting the wide dynamic range of biotin concentrations that are typically found in actual patient samples (i.e., 0-1500 ng/ml).

There are also other methods currently available to mitigate the problem of biotin interference. One method includes the use of a preformed reagent; that is, a biotinylated assay component is “pre-bound” to the streptavidin-coated solid support during reagent production and prior to interaction with the patient sample. Because of the tight binding and slow off-rate between streptavidin and biotin, the possibility that the biotin reagent already bound to streptavidin will be replaced by the incoming biotin in a patient sample is not a predominate process. The second method is to increase the streptavidin binding sites on the solid support, so that there are extra binding sites available for the sample biotin molecules in addition to the biotinylated assay components. The third method is the combination of both of the above. However, none of these three strategies truly solves the problem of biotin interference unless the use of biotin-streptavidin as active assay components is completely avoided. Another major issue with all of the above solutions is that assay components are involved in the prevention of interference, and this can easily affect the magnitude of the assay signal itself.

U.S. Pat. No. 5,212,063 refers to the use of polymer particles with a biotin binding core and a covering layer of protein, carbohydrate, or co-polymer for the purpose of filtering free biotin, but does not filter biotin conjugated to large molecules. However, U.S. Pat. No. 5,212,063 refers to the immobilization of streptavidin or other biotin binding molecule directly to the solid phase core to provide at least 10 binding sites for said free biotin or derivative of free biotin.

There is a need in the art for new and improved reagents that selectively remove free biotin from patient samples without significantly binding to the biotin moieties of assay reagents and without significantly interfering with the diagnostic assays being performed. It is to such reagents and kits and microfluidics devices containing same, as well as methods of producing and using same, that the present disclosure is directed.

### SUMMARY

The present disclosure provides compositions, kits, devices, and methods for selectively removing free biotin from biological samples.

In embodiments, the present disclosure includes a biotin-trap composition, including: a particle having an outer surface; an inner dextran aldehyde polymer coating layer disposed on at least a portion of the outer surface of the particle; a biotin-specific binding partner conjugated to the inner dextran aldehyde polymer coating layer, wherein the biotin-specific binding partner is avidin or an analog thereof; and at least one outer dextran aldehyde polymer coating layer disposed about the biotin-specific binding partner.

In embodiments, the present disclosure includes a kit including one or more biotin-trap compositions of the present disclosure; and at least one assay reagent for detecting the presence and/or concentration of a target analyte in a biological sample.

In embodiments, the present disclosure includes a microfluidics device for determining the concentration of at least one target analyte in a sample, the microfluidics device including: (i) an inlet channel through which a sample is applied; and (ii) at least one compartment capable of being in fluidic communication with the inlet channel, wherein the at least one compartment contains: (a) one or more biotin-trap compositions of the present disclosure; and (b) at least one assay reagent that includes biotin.

In embodiments, the present disclosure includes a method of producing a biotin-trap composition, the method including the steps of: (1) obtaining a particle having an outer surface with functional groups thereon; (2) reacting the particle with dextran aldehyde polymer under conditions that form an inner dextran aldehyde polymer coating layer disposed on at least a portion of the outer surface of the particle; (3) reacting the particle with a biotin-specific binding partner under conditions that conjugate the biotin-specific binding partner to the inner dextran aldehyde polymer coating layer, wherein the biotin-specific binding partner is avidin or an analog thereof; (4) reacting the particle with tetraethylene pentaamine (TPA) or poly(ethylene amine), (where n is 1 to 20); and (5 reacting the particle with dextran aldehyde polymer under conditions that form an outer dextran aldehyde polymer coating layer. In embodiments, steps (1-5) are performed in sequential order.

In embodiments, the present disclosure includes a method of substantially removing free biotin from a biological sample, the method including the step of: contacting the biological sample with the biotin-trap composition of the present disclosure; wherein the biological sample is contacted with the biotin-trap composition under conditions that allow free biotin present in the biological sample to bind or substantially bind to the biotin-trap composition.

In embodiments, the present disclosure includes a method of performing an assay for detecting the concentration of at least one target analyte in a biological sample, the method including the steps of: (1) contacting, either simultaneously or wholly or partially sequentially: the biological sample; the biotin-trap composition of the present disclosure; and at least one biotin-containing assay reagent; wherein the biological sample is contacted with the biotin-trap composition under conditions wherein the biotin-trap composition specifically binds to free biotin present in the biological sample but does not substantially bind to the biotin-containing assay reagent; (2) performing the assay using the at least one biotin-containing assay reagent; and (3) determining the concentration of the at least one target analyte in the biological sample.

### BRIEF DESCRIPTION OF THE DRAWINGS

FIG. 1 schematically depicts a synthesis scheme for production of one non-limiting embodiment of a biotin-trap composition in accordance with the present disclosure. TSTU: N, N, N′, N′-Tetramethyl-O—(N-succinimidyl) uronium tetrafluoroborate.

FIG. 2 schematically depicts the three-dimensional structure of biotin binding sites on streptavidin, as well as the acidic and basic amino acid residues adjacent to the biotin binding site and to which tetraethylene pentaamine (TPA) and dextran aldehyde are conjugated, in accordance with the present disclosure.

FIGS. 3A-3C schematically depict biotin-trap compositions constructed in accordance with the present disclosure.

FIG. 4 illustrates the use of biotin-trap compositions of the present disclosure in a TSH LOCI® assay. The results shown therein demonstrate the selective binding of free biotin by one of the biotin-trap compositions, thereby substantially reducing biotin interference in the assay.

FIG. 5 illustrates the use of biotin-trap compositions of the present disclosure in a TNIH LOCI® assay. The results shown therein demonstrate the selective binding of free biotin by one of the biotin-trap compositions, thereby substantially reducing biotin interference in the assay.

FIG. 6 illustrates the use of embodiments of biotin-trap compositions of the present disclosure in an FT3 LOCI® assay. The results shown therein demonstrate the selective binding of free biotin by one of the biotin-trap compositions, thereby substantially reducing biotin interference in the assay.

FIG. 7 illustrates the use of embodiments of biotin-trap compositions of the present disclosure in an FT4 LOCI® assay. The results shown therein demonstrate the selective binding of free biotin by one of the biotin-trap compositions, thereby substantially reducing biotin interference in the assay.

FIG. 8 illustrates the use of one embodiment of one non-limiting embodiment of biotin-trap composition to reduce biotin interference in a TSH LOCI® assay.

FIG. 9 illustrates the use of one embodiment of one non-limiting embodiment of biotin-trap composition to reduce biotin interference in an FT3 LOCI® assay.

FIG. 10 illustrates the use of one embodiment of one non-limiting embodiment of biotin-trap composition to reduce biotin interference in an FT4 LOCI® assay.

FIG. 11 depicts an exemplary block diagram of a computer system 1100.

FIG. 12 depicts a cross-sectional view of a biotin-trap composition embodiment in accordance with the present disclosure.

FIG. 13 depicts a schematic cross-sectional view of a microfluidic device of the present disclosure.

To facilitate understanding, identical reference numerals have been used, where possible, to designate identical elements that are common to the figures. The figures are not drawn to scale and may be simplified for clarity. Elements and features of one embodiment may be beneficially incorporated in other embodiments without further recitation.

### DETAILED DESCRIPTION

The present disclosure relates to one or more reagents that selectively remove free biotin from patient samples without significantly binding to the biotin moieties of assay reagents and/or without significantly interfering with the diagnostic assays being performed. In embodiments, the present disclosure includes reagents, kits and microfluidics devices containing same, as well as methods of producing and using same. In embodiments, the present disclosure relate to a biotin-trap composition, including: a particle having an outer surface; an inner dextran aldehyde polymer coating layer disposed on at least a portion of the outer surface of the particle; a biotin-specific binding partner conjugated to the inner dextran aldehyde polymer coating layer, wherein the biotin-specific binding partner is avidin or an analog thereof; and at least one outer dextran aldehyde polymer coating layer disposed about the biotin-specific binding partner. Moreover, the present disclosure relates to a microfluidics device including one or more biotin-trap compositions of the present disclosure. Advantages of the present disclosure include reducing or eliminating free biotin from patient samples without significantly binding to the biotin moieties of assay reagents and/or without significantly interfering with the diagnostic assays being performed.

Before describing additional embodiments of the present disclosure in detail by way of exemplary language and results, it is to be understood that the present disclosure is not limited in its application to the details of construction and the arrangement of the components set forth in the following description. The present disclosure is capable of other embodiments or of being practiced or carried out in various ways. As such, the language used herein is intended to be given the broadest possible scope and meaning; and the embodiments are meant to be exemplary-not exhaustive. Also, it is to be understood that the phraseology and terminology employed herein is for the purpose of description and should not be regarded as limiting.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The nomenclatures utilized in connection with, and the laboratory procedures and techniques of, analytical chemistry, synthetic organic chemistry, and medicinal and pharmaceutical chemistry described herein are those well-known and commonly used in the art. Unless otherwise stated, standard techniques are used for chemical syntheses and chemical analyses.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

All of the articles, compositions, kits, devices, and/or methods disclosed herein can be made and executed without undue experimentation in light of the present disclosure. While the articles, compositions, kits, devices, and/or methods have been described in terms of particular embodiments, it will be apparent to those of skill in the art that variations may be applied to the articles, methods compositions, kits, and/or and in the steps or in the sequence of steps of the methods described herein without departing from the concept, spirit, and scope of the present disclosure. All such similar substitutes and modifications apparent to those skilled in the art are deemed to be within the spirit, scope, and concept of the present disclosure as defined by the appended claims.

### Definitions

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. The term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the phrases “associated with” and “coupled to” include both direct association/binding of two moieties to one another as well as indirect association/binding of two moieties to one another. Non-limiting examples of associations/couplings include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example.

The terms “analog” and “derivative” are used herein interchangeably and refer to a substance which comprises the same basic carbon skeleton and carbon functionality in its structure as a given compound, but can also contain one or more substitutions thereto. The term “substitution” as used herein will be understood to refer to the replacement of at least one substituent on a compound with a residue R. In certain non-limiting embodiments, R may include H, hydroxyl, thiol, a halogenid selected from fluoride, chloride, bromide, or iodide, a C1-C4 compound selected one of the following: linear, branched or cyclic alkyl, optionally substituted, and linear branched or cyclic alkenyl, wherein the optional substitutents are selected from one or more alkenylalkyl, alkynylalkyl, cycloalkyl, cycloalkenylalkyl, arylalkyl, heteroarylalkyl, heterocyclealkyl, optionally substituted heterocycloalkenylalkyl, arylcycloalkyl, and arylheterocycloalkyl, each of which is optionally substituted wherein the optional substitutents are selected from one or more of alkenylalkyl, alkynylalkyl, cycloalkyl, cyclalkenylalkyl, arylalkyl, alkylaryl, heteroarylalkyl, heterocyclealkyl, optionally substituted heterocycloalkenylalkyl, arylcycloalkyl, and arylheterocyclalkyl, phenyl, cyano, hydroxyl, alkyl, aryl, cycloalkyl, cyano, alkoxy, alkylthio, amino, —NH (alkyl), —NH(cycloalkyl)₂, carboxy, and —C(O))-alkyl.

The term “sample” as used herein will be understood to include any type of biological sample that may be utilized in accordance with the present disclosure. Examples of fluidic biological samples that may be utilized include, but are not limited to, whole blood or any portion thereof (i.e., plasma or serum), urine, saliva, sputum, cerebrospinal fluid (CSF), skin, intestinal fluid, intraperitoneal fluid, cystic fluid, sweat, interstitial fluid, extracellular fluid, tears, mucus, bladder wash, semen, fecal, pleural fluid, nasopharyngeal fluid, combinations thereof, and the like.

The term “specific binding partner,” as used in particular (but not by way of limitation) herein in the terms “biotin-specific binding partner” or “target analyte-specific binding partner,” will be understood to refer to any molecule capable of specifically associating with biotin or the target analyte, respectively. For example, but not by way of limitation, the binding partner may be an antibody, a receptor, a ligand, aptamers, molecular imprinted polymers (e.g., inorganic matrices), combinations or derivatives thereof, as well as any other molecules capable of specific binding to biotin or the target analyte, respectively.

The term “antibody” is used herein in the broadest sense and refers to an agent that specifically binds to a particular antigen. In some embodiments, the term encompasses any polypeptide or polypeptide complex that includes immunoglobulin structural elements sufficient to confer specific binding. Such polypeptide may be naturally produced (e.g., generated by an organism reacting to an antigen), or produced by recombinant engineering, chemical synthesis, or other artificial system or methodology. Non-limiting examples include intact monoclonal antibodies and polyclonal antibodies, multi-specific antibodies (e.g., bispecific antibodies), antibody fragments and conjugates thereof that exhibit the desired biological activity of analyte binding (such as, but not limited to, Fab, Fab′, F(ab′)2, Fv, scFv, Fd, diabodies, single-chain antibodies, and other antibody fragments and conjugates thereof that retain at least a portion of the variable region of an intact antibody), antibody substitute proteins or peptides (i.e., engineered binding proteins/peptides), and combinations or derivatives thereof. The antibody can be of any type or class (e.g., IgG, IgE, IgM, IgD, and IgA) or sub-class (e.g., IgG1, IgG2, IgG3, IgG4, IgA1, and IgA2). In some embodiments, an antibody may contain a covalent modification (e.g., attachment of a glycan, a payload (e.g., a detectable moiety, a therapeutic moiety, a catalytic moiety, etc.), or other pendant group (e.g., poly-ethylene glycol, etc.).

An “analyte” is a macromolecule that is capable of being recognized by an analyte-specific binding partner, such as (but not limited to) an antibody. Both analytes and haptens include at least one antigenic determinant or “epitope,” which is the region of the antigen or hapten which binds to the analyte-specific binding partner (e.g., antibody). Typically, the epitope on a hapten is the entire molecule.

The term “detection agent” as used herein refers to any element, molecule, functional group, compound, fragment or moiety that is detectable. In some embodiments, a detection agent is provided or utilized alone. In some embodiments, a detection agent is provided and/or utilized in association with (e.g., joined to) another agent. Examples of detection agents include, but are not limited to: various ligands, radionuclides (e.g., ³H, ¹⁴C, ¹⁸F, ¹⁹F, ³²P, ³⁵S, ¹³⁵I, ¹²⁵I, ¹²³I, ⁶⁴Cu, ¹⁸⁷Re, ¹¹¹In, ⁹⁰Y, ^{99m}Tc, ¹⁷⁷Lu, ⁸⁹Zr etc.), fluorescent dyes, chemiluminescent agents (such as, for example, acridinum esters, stabilized dioxetanes, and the like), bioluminescent agents, spectrally resolvable inorganic fluorescent semiconductors nanocrystals (i.e., quantum dots), metal nanoparticles (e.g., gold, silver, copper, platinum, etc.) nanoclusters, paramagnetic metal ions, enzymes, colorimetric labels (such as, for example, dyes, colloidal gold, and the like), biotin, dioxigenin, haptens, and proteins for which antisera or monoclonal antibodies are available.

As used herein, “diagnostic test” is a step or series of steps that is or has been performed to attain information that is useful in determining whether a patient has a disease, disorder or condition and/or in classifying a disease, disorder or condition into a phenotypic category or any category having significance with regard to prognosis of a disease, disorder or condition, or likely response to treatment (either treatment in general or any particular treatment) of a disease, disorder or condition. Similarly, “diagnosis” refers to providing any type of diagnostic information, including, but not limited to, whether a subject is likely to have or develop a disease, disorder or condition, state, staging or characteristic of a disease, disorder or condition as manifested in the subject, information related to the nature or classification of a tumor, information related to prognosis and/or information useful in selecting an appropriate treatment or additional diagnostic testing. Selection of treatment may include the choice of a particular therapeutic agent or other treatment modality such as surgery, radiation, etc., a choice about whether to withhold or deliver therapy, a choice relating to dosing regimen (e.g., frequency or level of one or more doses of a particular therapeutic agent or combination of therapeutic agents), etc. Selection of additional diagnostic testing may include more specific testing for a given disease, disorder, or condition.

The term “LOCI®” as used herein refers to the Luminescent Oxygen Channeling Assay technology. The LOCI® advanced chemiluminescence assay is described, for example, in U.S. Pat. No. 5,340,716 (Ullman et al.), the entire contents of which are expressly incorporated herein by reference. The currently available LOCI® technology has high sensitivity and uses several reagents. In particular, the LOCI® assay requires that two of these reagents (referred to as a “sensibead” and a “chemibead”) be held by other specific binding partner assay reagents in a manner whereby the sensibead and chemibead are in close proximity to one another to achieve a signal. Upon exposure to light at a certain or predetermined wavelength, the sensibead releases singlet oxygen, and if the two beads are in close proximity, the singlet oxygen is transferred to the chemibead; this causes a chemical reaction that results in the chemibead giving off light that can be measured at a different wavelength.

As used herein, the term “subject” refers to an organism, for example, a mammal (e.g., a human). In some embodiments a human subject is an adult, adolescent, or pediatric subject. In some embodiments, a subject is at least 50, at least 55, at least 60, at least 65, at least 70, at least 75, or at least 80 years of age. In some embodiments, a subject is suffering from a disease, disorder or condition, e.g., a disease, disorder or condition that can be treated as provided herein. In some embodiments, a subject is susceptible to a disease, disorder, or condition; in some embodiments, a susceptible subject is predisposed to and/or shows an increased risk (as compared to the average risk observed in a reference subject or population) of developing the disease, disorder or condition. In some embodiments, a subject displays one or more symptoms of a disease, disorder or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a patient. In some embodiments, a subject is an individual to whom diagnosis and/or therapy is and/or has been administered.

Threshold value: As used herein, the term “threshold value” refers to a value (or values) that are used as a reference to attain information on and/or classify the results of a measurement, for example, the results of a measurement attained in an assay. A threshold value can be determined based on one or more control samples. A threshold value can be determined prior to, concurrently with, or after the measurement of interest is taken. In some embodiments, a threshold value can be a range of values. In some embodiments, a threshold value can be a value (or range of values) reported in the relevant field (e.g., a value found in a standard table).

Turning now to certain embodiments of the present disclosure, certain non-limiting embodiments of the present disclosure are directed to one or more biotin-trap compositions. The biotin-trap compositions include a particle having an outer surface; an inner dextran aldehyde polymer coating layer disposed on at least a portion of the outer surface of the particle; and a biotin-specific binding partner conjugated to the inner dextran aldehyde polymer coating layer, wherein the biotin-specific binding partner is avidin or an analog thereof.

In embodiments, the biotin-trap compositions further include at least one outer dextran aldehyde polymer coating layer disposed about the biotin-specific binding partner. In addition, the biotin-trap compositions may further include tetraethylene pentaamine (TPA) or poly(ethylene amine), (where n is 1 to 20) that binds to the biotin-specific binding partner and retains the at least one outer dextran aldehyde polymer coating layer disposed about (and associated with) the biotin-specific binding partner.

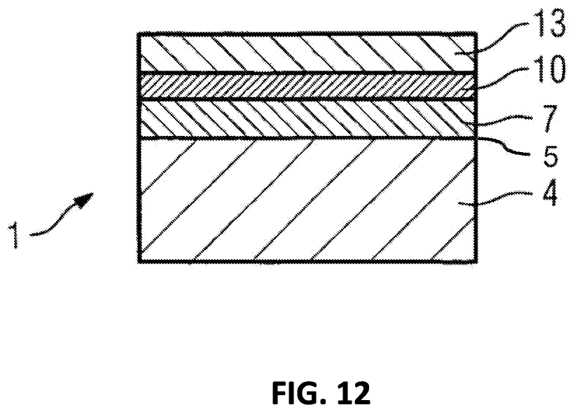

Referring now to FIG. 12 a cross-sectional view of a portion of a biotin-trap compositions is shown. More specifically, FIG. 12 depicts a biotin-trap composition 1, including: a particle 4 having an outer surface 5. An inner dextran aldehyde polymer coating layer 7 is shown disposed on at least a portion of the outer surface 5 of the particle 4. In embodiments, the inner dextran aldehyde polymer coating layer 7 is provided in an amount suitable to continuously coat particle 4 and the entire outer surface 5. In embodiments, the thickness of the inner dextran aldehyde polymer coating layer 7 is predetermined. A biotin-specific binding partner 10 is shown conjugated to the inner dextran aldehyde polymer coating layer 7. In embodiments, the biotin-specific binding partner 10 is avidin or an analog thereof. In embodiments, the biotin-specific binding partner 10 is provided in an amount suitable to continuously coat the inner dextran aldehyde polymer coating layer 7. However, in embodiments, the biotin-specific binding partner 10 is provided in an amount suitable to cover portions of the inner dextran aldehyde polymer coating layer 7. At least one outer dextran aldehyde polymer coating layer 13 is shown disposed about the biotin-specific binding partner 10. In embodiments, the outer dextran aldehyde polymer coating layer 13 is disposed atop the biotin-specific binding partner 10 in a continuous layer. In embodiments, the biotin-trap composition includes tetraethylene pentaamine (TPA) or poly(ethylene amine) n (where n is 1 to 20) that binds to the biotin-specific binding partner 10 and retains the at least one outer dextran aldehyde polymer coating layer 13 disposed about the biotin-specific binding partner 10. In embodiments, the particle 4 is selected from the group consisting of a polystyrene bead, a latex bead, a magnetic particle, a non-magnetic particle, or combinations thereof. In embodiments, the outer surface 5 of the particle 4 has functional groups thereon for associating with the dextran aldehyde polymer. In embodiments, the particle 4 includes a polystyrene bead having carboxylate functional groups on the outer surface thereof. In embodiments, the biotin-specific binding partner 10 is streptavidin.

In biotin-trap composition embodiments of the present disclosure, basic amino acid residues are contacted with Dexal, and acidic side chains are contacted with TPA or poly(ethylene amine), (where n is 1 to 20), which is then conjugated and covered by another layer of Dexal. Some of these charged side chains are adjacent or near the biotin binding sites. Therefore, in embodiments, suitable conditions are provided such that both the acidic and basic amino acid residues near the biotin binding sites are conjugated to Dexal polymers so to provide a better steric hindrance for biotin conjugated to large protein molecules, while allowing free biotin to filter through.

In some embodiments, the present disclosure includes a biotin-trap composition, including: a particle having an outer surface; an inner dextran aldehyde polymer coating layer disposed on at least a portion of the outer surface of the particle; a biotin-specific binding partner conjugated to the inner dextran aldehyde polymer coating layer, wherein the biotin-specific binding partner is avidin or an analog thereof; at least one outer dextran aldehyde polymer coating layer disposed about the biotin-specific binding partner; and tetraethylene pentaamine (TPA) or poly(ethylene amine), (where, is 1 to 20) that binds to the biotin-specific binding partner and retains the at least one outer dextran aldehyde polymer coating layer disposed about the biotin-specific binding partner.

In some embodiments, the present disclosure includes a biotin-trap composition, including: a particle having an outer surface; an inner dextran aldehyde polymer coating layer disposed on at least a portion of the outer surface of the particle; a biotin-specific binding partner conjugated to the inner dextran aldehyde polymer coating layer, wherein the biotin-specific binding partner is avidin or an analog thereof; and at least one outer dextran aldehyde polymer coating layer disposed about the biotin-specific binding partner; wherein the particle is selected from the group consisting of a polystyrene bead, a latex bead, a magnetic particle, or a non-magnetic particle, and combinations thereof. In embodiments, the outer surface of the particle has functional groups thereon for associating with the dextran aldehyde polymer. In embodiments, the particle includes a polystyrene bead having carboxylate functional groups on the outer surface thereof.

In some embodiments, the present disclosure includes a biotin-trap composition, including: a particle having an outer surface; an inner dextran aldehyde polymer coating layer disposed on at least a portion of the outer surface of the particle; a biotin-specific binding partner conjugated to the inner dextran aldehyde polymer coating layer, wherein the biotin-specific binding partner is avidin or an analog thereof; and at least one outer dextran aldehyde polymer coating layer disposed about the biotin-specific binding partner; wherein the biotin-specific binding partner is streptavidin.

Any particles known in the art or otherwise contemplated herein may be utilized in accordance with the present disclosure, so long as the biotin-trap compositions formed therefrom can function to specifically bind free biotin and not substantially bind or bind any biotin-containing assay reagents (and thus allow these conjugates to be filtered out). For example (but not by way of limitation, the particles can be polystyrene beads, latex beads, magnetic particles, non-magnetic particles, and the like. In particular (but non-limiting) embodiments, the particles utilized in the biotin-trap compositions are the same or similar to the particles utilized in the assay; in this manner, no issues will arise with differences in specific gravity, particle stability, etc. between the biotin-trap compositions and the particle reagents utilized in the assay.

In embodiments, the particles are provided with (or modified to have) functional groups on the outer surface thereof that associate with the dextran aldehyde polymer and thus form the inner Dexal polymer coating layer. For example, but not by way of limitation, the particles may be polystyrene beads having functional groups (such as, but not limited to, aldehyde functional groups, carboxylate functional groups, amide functional groups (such as, but not limited to, amides of poly(ethylene amine)), and alkyl hydrazine functional groups) thereon. Then the particles may be further reacted with another reactant; for example, but not by way of limitation, the particles having aldehyde or carboxylate functional groups may be further reacted with hydrazine to form a hydrazone.

Any biotin-specific binding partners known in the art or otherwise contemplated herein may be utilized in accordance with the present disclosure. In certain non-limiting embodiments, the biotin-specific binding partner is avidin or an analog thereof. Particular non-limiting examples of avidin analogs include avidin, streptavidin, traptavidin, neutral avidin, Neutralite avidin, Neutravidin, Lite-avidin, succinylated avidin, other forms of modified or genetically engineered) avidin, esters, salts, and/or derivatives of any of the above, and the like, or combinations thereof.

Certain non-limiting embodiments of the present disclosure are directed to kits that contain one or more of any of the biotin-trap compositions disclosed or otherwise contemplated herein. In certain particular (but non-limiting) embodiments, the kit further includes at least one assay reagent for detecting the presence and/or concentration of a target analyte in a biological sample.

In certain particular (but non-limiting) embodiments, the at least one assay reagent may be a biotin-containing assay reagent. One non-limiting example of such a reagent is a biotinylated target analyte-specific binding partner, such as (but not limited to) a biotinylated antibody.

In certain particular (but non-limiting) embodiments, the at least one assay reagent present in the kit may be at least one assay reagent utilized in a chemiluminescent detection system (such as, but not limited to, a LOCI® assay system) for determining the concentration of at least one target analyte in a sample. Non-limiting examples thereof include one or more of the following: (a) a composition including a singlet oxygen-activatable chemiluminescent compound having an analyte-specific binding partner for a target analyte directly or indirectly bound thereto, as well as a fluorescent molecule that is excited by the activated chemiluminescent compound; (b) a biotinylated analyte-specific binding partner for the target analyte; and (c) a composition including a sensitizer capable of generating singlet oxygen in its excited state and having a biotin-specific binding partner directly or indirectly bound thereto.

Any target analyte-specific binding partners known in the art or otherwise contemplated herein may be utilized in accordance with the present disclosure. Non-limiting examples of analyte-specific binding partners for target analytes include an antibody, a receptor, a ligand, an aptamer, a molecular imprinted polymer (i.e., inorganic matrix), and any combinations or derivatives thereof, as well as any other molecules capable of specific binding to the target analyte.

The compositions/reagents of the kits may be provided in any form that allows them to function in accordance with the present disclosure. For example, but not by way of limitation, each of the reagents may be provided in liquid form and disposed in bulk and/or single aliquot form within the kit. Alternatively, in a particular (but non-limiting) embodiment, one or more of the reagents may be disposed in the kit in the form of a single aliquot lyophilized reagent. The use of dried reagents in kits/microfluidics devices is described in detail in U.S. Pat. No. 9,244,085 (Samproni), the entire contents of which are hereby expressly incorporated herein by reference.

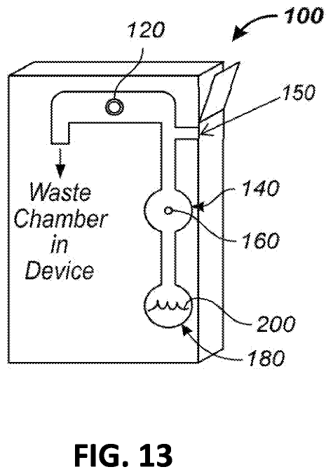

Referring now to FIG. 13, FIG. 13 depicts a kit embodiments such as a microfluidic device 100 suitable for use in accordance with the present disclosure. In embodiments, microfluidic device 100 is suitable for use in monitoring analytes of interest in a biological sample. In embodiments, the microfluidic device 100 may be any type of disposable cartridge (for example but not by way of limitation, a laminate card or a molded card) or the like, and capable of containing a microfluidic structure as described herein. In embodiments, the microfluidic device 100 contains a sensor 120 and elements for producing one or more solution(s) for calibration and/or quality control. These elements include at least one compartment 140 in fluidic communication with the sensor 120. In embodiments, the compartment 140 contains at least one lyophilized reagent 160 (the lyophilized reagent depicted in bead/hemisphere form and/or may include one or more biotin-trap compositions of the present disclosure. The compartment 140 is also in fluidic communication with an activatable cavity 180 containing an excipient 200 (such as but not limited to, a liquid or a gel) for reconstitution of the lyophilized reagent 160 (wherein the fluidic communication is provided upon activation of the activatable cavity 180). The activatable cavity 180 may be, for example but not by way of limitation, a blister pack or other type of sealed cavity that is sealed to prevent contact between the excipient 200 and the lyophilized reagent 160 until use of the microfluidic device 100. In use, the activatable cavity 180 may be activated such as (but not by way of limitation) by depression thereof, thus pushing the excipient 200 into the compartment 140 containing the lyophilized reagent 160 to reconstitute the lyophilized reagent 160, thereby providing a reconstituted calibration and/or quality control solution. The reconstituted calibration and/or quality control solution is then brought into contact with the sensor 120. The resultant reconstituted calibration and/or quality control solution will contain a known quantity of ions, proteins and/or gases that will serve as calibrators and/or control solutions for the sensor of interest.

In certain embodiments, some mixing may occur in the compartment 140 between the lyophilized reagent 160 and the excipient 200 to ensure complete reconstitution of the lyophilized reagent 160 and to ensure homogeneity of the resultant reconstituted calibration and/or quality control solution. Any method of mixing known in the microfluidics art or otherwise contemplated herein may be utilized in accordance with the presently disclosed and claimed inventive concept(s). In addition, the flow of the excipient 200 into the compartment 140 may be controlled by any method known in the art or otherwise contemplated herein; for example, but not by way of limitation, the force of activating (i.e., depressing) the activatable cavity 180 may provide the necessary force to push the desired amount of excipient 200 into the compartment 140. Likewise, the flow of the reconstituted calibration and/or quality control solution (whether in the form of ion(s), protein(s) and/or gas(es)) from the compartment 140 and over the sensor 120 may be controlled by any method known in the art or otherwise contemplated herein; for example but not by way of limitation, the force of activating (i.e., depressing) the activatable cavity 180 may ultimately provide the necessary force to push the reconstituted calibration and/or quality control solution out of the compartment 140 and over the sensor 120.

In embodiments, a microfluidics device 100 is provided for determining the concentration of at least one target analyte in a sample, the microfluidics device including: (i) an inlet channel 150 through which a sample is applied; and (ii) at least one compartment capable of being in fluidic communication with the inlet channel 15), wherein the at least one compartment contains: (a) the biotin-trap composition of the present disclosure; and (b) at least one assay reagent that includes biotin. In embodiments, the at least one assay reagent is for use in a chemiluminescent detection system. In embodiments, the at least one assay reagent is a biotinylated target analyte-specific binding partner. In embodiments, the biotinylated target analyte-specific binding partner is a biotinylated antibody. In embodiments, the microfluidics device 100 is configured for insertion in an automated diagnostic test instrument system.

In addition to the compositions/reagents described in detail herein above, the kits may further contain other reagent(s) for conducting any of the particular assays described or otherwise contemplated herein. The nature of these additional reagent(s) will depend upon the particular assay format, and identification thereof is well within the skill of one of ordinary skill in the art; therefore, no further description thereof is deemed necessary. Also, the compositions/reagents present in the kits may each be in separate containers/compartments, or various compositions/reagents can be combined in one or more containers/compartments, depending on the cross-reactivity and stability of the compositions/reagents. In addition, the kit may include a microfluidics device in which the compositions/reagents are disposed.

The relative amounts of the various compositions/reagents in the kits can vary widely to provide for concentrations of the compositions/reagents that substantially optimize the reactions that need to occur during the assay methods and further to optimize substantially the sensitivity and selectivity of an assay. Under appropriate circumstances, one or more of the compositions/reagents in the kit can be provided as a dry powder, such as a lyophilized powder, and the kit may further include excipient(s) for dissolution of the dried reagents; in this manner, a reagent solution having the appropriate concentrations for performing a method or assay in accordance with the present disclosure can be obtained from these compositions. Positive and/or negative controls may also be included with the kit. In addition, the kit can further include a set of written instructions explaining how to use the kit. A kit of this nature can be used in any of the methods described or otherwise contemplated herein.

Certain additional non-limiting embodiments of the present disclosure are directed to a microfluidics device that includes the compositions of any of the kits described herein above. In particular, certain non-limiting embodiments include a microfluidics device for determining the concentration of at least one target analyte in a sample. The microfluidics device includes (i) an inlet channel through which a sample is applied; and (ii) at least a first compartment capable of being in fluidic communication with the inlet channel. The compartment(s) of (ii) contains: (a) any of the biotin-trap compositions disclosed or otherwise contemplated herein; and (b) at least one assay reagent that specifically binds to the at least one target analyte. Any of the assay reagents disclosed or otherwise contemplated herein may be utilized in the microfluidics devices of the present disclosure.

The microfluidics device may be provided with any arrangement of the compartments and distribution of the various compositions therebetween that allows the device to function in accordance with the present disclosure. That is, the biotin-trap composition and the at least one assay reagent may be disposed in the same compartment or in different compartments. When the two reagents are separated between two compartments, the biotin-trap composition may be disposed in a first compartment that is in fluidic communication with the inlet channel, and the at least one assay reagent may be disposed in a second compartment that is in fluidic communication with the first compartment. In this manner, the biotin-trap composition may encounter the sample first and substantially removes free biotin therefrom prior to contacting the sample with the at least one assay reagent; alternatively, the sample may contact the at least one assay reagent prior to the biotin-trap composition.

In certain particular (but non-limiting) embodiments, the microfluidics device may be configured for insertion into an automated diagnostic test instrument system that performs the diagnostic assay. Alternatively, the microfluidics device may be a standalone product that can be read without a diagnostic test instrument system.

Any of the compartments of the microfluidics device may be sealed to maintain reagent(s) disposed therein in a substantially airtight environment until use thereof; for example, compartments containing lyophilized reagent(s) may be sealed to prevent any unintentional reconstitution of the reagent. The inlet channel and a compartment, as well as two compartments, may be described as being “capable of being in fluidic communication” with one another; this phrase indicates that each of the compartment(s) may still be sealed, but that the two compartments are capable of having fluid flow therebetween upon puncture of a seal formed therein or therebetween.

The microfluidics devices of the present disclosure may be provided with any other desired features known in the art or otherwise contemplated herein. For example, but not by way of limitation, the microfluidics devices of the present disclosure may further include a read chamber; the read chamber may be any of the compartments containing the reagents described herein above, or the read chamber may be in fluidic communication with said compartment. The microfluidics device may further include one or more additional compartments containing other solutions, such as (but not limited to) wash solutions, dilution solutions, excipients, interference solutions, positive controls, negative controls, quality controls, and the like. These additional compartment(s) may be in fluidic communication with one or more of the other compartments. For example, the microfluidics device may further include one or more compartments containing a wash solution, and these compartment(s) may be capable of being in fluidic communication with any other compartment(s) of the device. In another example, the microfluidics device may further include one or more compartments containing an excipient for dissolution of one or more dried reagents, and the compartment(s) may be capable of being in fluidic communication with any other compartment(s) of the device. In yet a further example, the microfluidics device may include one or more compartments containing a dilution solution, and the compartment(s) may be capable of being in fluidic communication with any other compartment(s) of the device.

Certain non-limiting embodiments of the present disclosure are directed to a method of producing any of the biotin-trap compositions disclosed or otherwise contemplated herein. In the method, a particle having functional groups thereon is reacting with dextran aldehyde polymer under conditions that form an inner dextran aldehyde polymer coating layer on at least a portion of the outer surface of the particle, then reacting the modified particle with a biotin-specific binding partner that conjugate the biotin-specific binding partner to the inner polymer coating layer. The method may further include reacting said particle with dextran aldehyde polymer under conditions that form an outer dextran aldehyde polymer coating layer.

In certain particular (but non-limiting) embodiments, the method includes the steps of: (1) obtaining a particle having an outer surface with carboxylate functional groups thereon; (2) reacting the particle with hydrazine; (3) reacting the particle with dextran aldehyde polymer under conditions that form an inner dextran aldehyde polymer coating layer disposed on at least a portion of the outer surface of the particle; and (4) reacting the particle with a biotin-specific binding partner under conditions that conjugate the biotin-specific binding partner to the inner dextran aldehyde polymer coating layer, wherein the biotin-specific binding partner is avidin or an analog thereof. In particular (but non-limiting) embodiments, the method further includes the steps of: (5) reacting the particle with tetraethylene pentaamine (TPA) or poly(ethylene amine), (where n is 1 to 20); and (6) reacting the particle with dextran aldehyde polymer under conditions that form an outer dextran aldehyde polymer coating layer. In embodiments, the steps (1)-(4), or (1)-(6) are performed in sequentially or in sequential order.

Certain non-limiting embodiments of the present disclosure are directed to a method of substantially removing free biotin from a biological sample. In the method, the biological sample is contacted with any of the biotin-trap compositions disclosed or otherwise contemplated herein under conditions that allow free biotin present in the biological sample to substantially bind to the biotin-trap composition.

Certain non-limiting embodiments of the present disclosure are directed to a method of performing an assay for detecting the concentration of at least one target analyte in a biological sample. The method includes the steps of: (1) contacting, either simultaneously or wholly or partially sequentially, the biological sample with any of the biotin-trap compositions disclosed or otherwise contemplated herein and at least one of any of the biotin-containing assay reagents disclosed or otherwise contemplated herein, under conditions whereby the biotin-trap composition specifically binds to free biotin present in the biological sample (thereby substantially removing free biotin from the biological sample) but does not substantially bind to the biotin-containing assay reagent; (2) performing the assay using the at least one biotin-containing assay reagent; and (3) determining the concentration of the at least one target analyte in the biological sample.

In certain particular (but non-limiting) embodiments, the assay is a chemiluminescent detection assay, such as (but not limited to), a LOCI® assay.

Non-limiting examples of biological samples that may be utilized in accordance with the various methods of the present disclosure include whole blood or any portion thereof (i.e., plasma or serum), urine, saliva, sputum, cerebrospinal fluid (CSF), skin, intestinal fluid, intraperitoneal fluid, cystic fluid, sweat, interstitial fluid, extracellular fluid, tears, mucus, bladder wash, semen, fecal, pleural fluid, nasopharyngeal fluid, and combinations thereof. Particular non-limiting examples include lysed whole blood cells and lysed red blood cells.

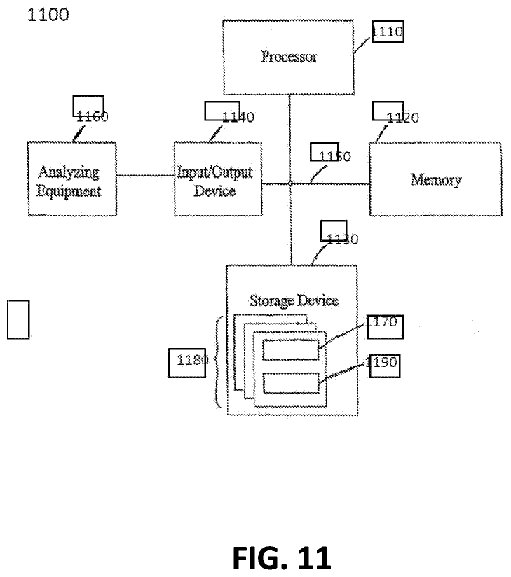

Referring now to FIG. 11 a block diagram of a computer system 1100 that can be used in the operations described above and methods of the present disclosure, according to one embodiment is shown. The system 1100 includes a processor 1110, a memory 1120, a storage device 1130 and an input/output device 1140. Each of the components 1110, 1120, 1130 and 1140 are interconnected using a system bus 1150. The system may include analyzing equipment 1160 for determining a level of free biotin in a sample or for performing a diagnostic assay. The processor 1110 is capable of processing instructions for execution within the system 1100. In one embodiment, the processor 1110 is a single-threaded processor. In another embodiment, the processor 1110 is a multi-threaded processor. The processor 1110 is capable of processing instructions stored in the memory 1120 or on the storage device 1130, including for receiving or sending information through the input/output device 1140. The memory 1120 stores information within the system 1100. In one embodiment, the memory 1120 is a computer-readable medium. In one embodiment, the memory 1120 is a volatile memory unit. In another embodiment, the memory 1120 is a non-volatile memory unit. In embodiments, the storage device 1130 is capable of providing mass storage for the system 1100. In one embodiment, the storage device 1130 is a computer-readable medium. The input/output device 1140 provides input/output operations for the system 1100. In one embodiment, the input/output device 1140 includes a keyboard and/or pointing device. Preferably, methods of the present disclosure are performed in a system 1100. For example, a computer program product can include instructions that cause a processor 1110 to perform the steps of a method of the present disclosure.

Additionally, non-transitory computer readable media containing executable instructions that when executed cause a processor to perform operations including a method as provided herein are provided. For example, a non-transitory computer readable medium containing executable instructions that when executed cause a processor to perform operations including a method as described herein such as a method of producing a biotin-trap composition, a method of substantially removing free biotin from a biological sample, and/or a method of performing an assay for detecting the concentration of at least one target analyte in a biological sample as described herein including the operations described for each method.

As mentioned above, the various compositions utilized in the methods are provided in combination (either simultaneously or sequentially). When the various compositions utilized in the method are added sequentially, the order of addition of the compositions may be varied; a person having ordinary skill in the art can determine the particular desired order of addition of the different compositions to the assay. The simplest order of addition, of course, is to add all the materials simultaneously and determine the signals produced therefrom. Alternatively, each of the compositions, or groups of compositions, can be combined sequentially. In certain embodiments, an incubation step may be involved subsequent to one or more additions.

While particular embodiments of the present disclosure are described as having the LOCI® assay format, it is to be understood that the present disclosure is also directed to other assay formats (and compositions, kits, microfluidics devices, and methods of performing same) for which elimination of sample biotin interference is desired. For example (but not by way of limitation), the present disclosure also includes assay formats where different signal molecules such as (but not limited to) different antibodies linked to different enzymes that generate signals at different wavelengths can be utilized in place of the chemiluminescent compound-containing compositions described herein above.

### Example

An Example is provided hereinbelow. However, the present disclosure is to be understood to not be limited in its application to the specific experimentation, results, and laboratory procedures disclosed herein. Rather, the Example is simply provided as one of various embodiments and are meant to be exemplary, not exhaustive.

Biotin interference is a major issue for in vitro diagnostics when assay reagents utilize biotin/streptavidin pairs. In the present Example, biotin-trap beads were produced that allow for the selective removal of free biotin from patient samples without significantly binding the biotin moiety present in assay reagent(s) (such as a biotinylated antibody) in the reaction mixture.

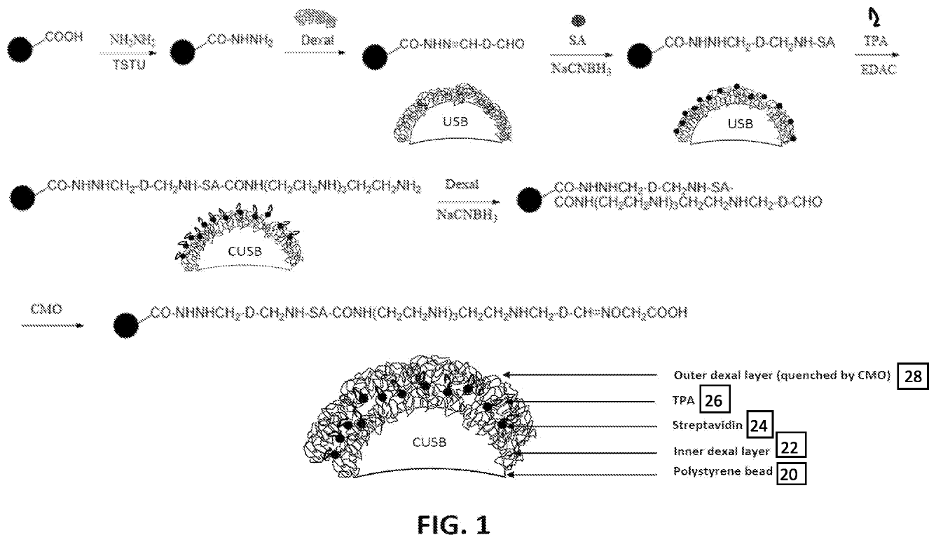

This Example describes the production of one non-limiting embodiment of biotin-trap beads constructed in accordance with the present disclosure. As shown in FIG. 1, a substrate such as one or more polystyrene beads with carboxylate functional groups were first reacted with hydrazine and then coated with dextran aldehyde polymer (referred to herein interchangeably as “Dexal,” “dexOx,” and “Dextran-CHO”), and then streptavidin molecules were added to react with the coated Dexal layer. The conjugated streptavidin layer was then reacted with tetraethylene pentaamine (TPA) and again Dexal to form one or more additional polymer layer(s) wrapped around streptavidin. The polymer layer(s) around streptavidin made the beads capable of selectively trapping free biotin from patient samples without binding the biotin-moieties conjugated to assay antibodies or proteins due to steric hindrance. This led to selective removal of free biotin in the reaction mixture and resulted in biotin interference mitigation. Referring now to FIG. 1, a substrate such as polystyrene bead (20) is shown including an inner dexal layer (22) disposed atop the polystyrene bead (20). Streptavidin (24) is shown atop inner dexal layer (22). TPA (26) is shown atop streptavidin (24), and outer dexal layer (28) is shown disposed atop TPA (26).

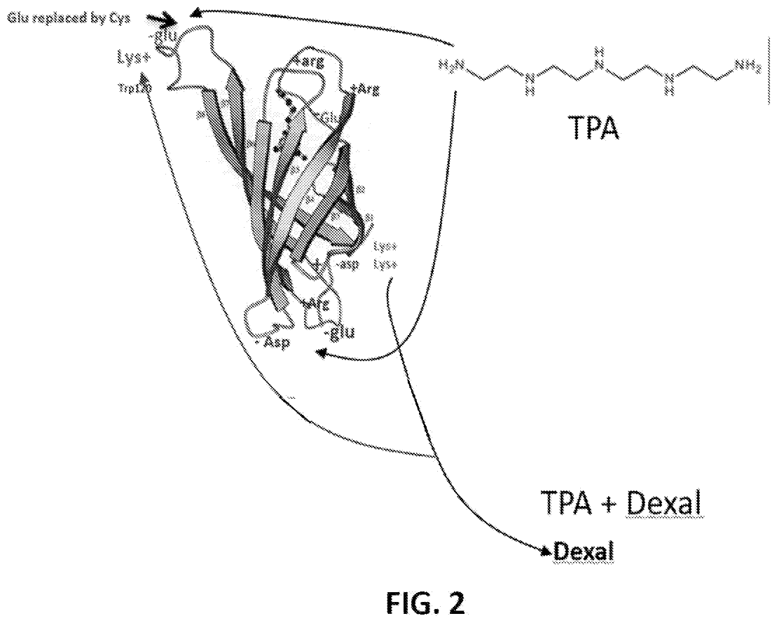

The present disclosure is the first composition including one or more biotin-traps that demonstrated significant selective free-biotin binding over conjugated biotin. In embodiments, at least 2-3 polymer layers or several polymer layers are included to densely wrap around the entrances of biotin at the binding sites in streptavidin. The densely wrapped entrances sterically hindered conjugated biotin binding but allowed the diffusion of free biotin into the binding sites. In particular, after streptavidin was conjugated to Dexal-coated beads, TPA (tetraethylene pentaamine) was added to functionalize the acidic amino acid residues near the entrances of biotin, which allowed more condensed covering of the entrances with Dexal. The 3-D structure of biotin binding sites on streptavidin as well as acidic and basic amino acid residues near the biotin entrances is shown in FIG. 2, and TPA and Dexal conjugations to the acidic and basic amino acid residues are illustrated in FIG. 2.

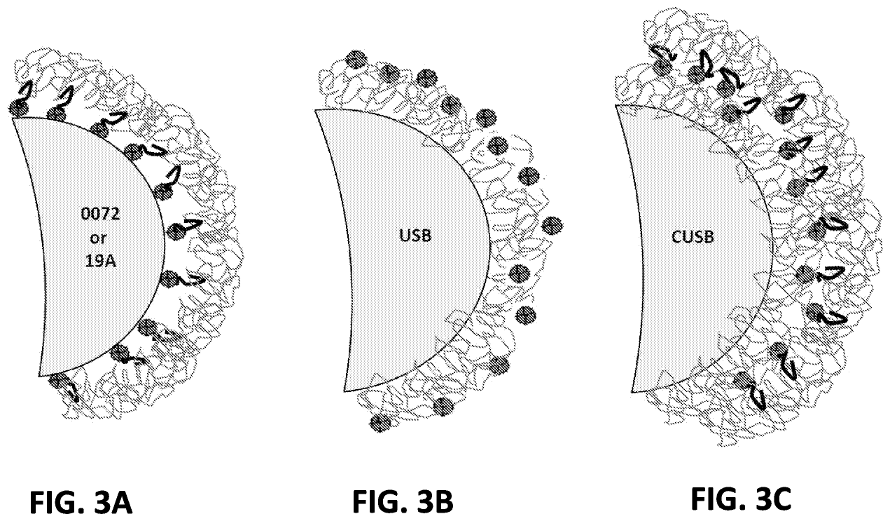

Diagrams of three different embodiments of biotin-trap compositions produced are shown in FIGS. 3A-3C, respectively. The bead shown in the right panel, FIG. 3C, was produced by the synthesis scheme of FIG. 1. The bead in the middle panel (FIG. 3B) was prepared using only the first three synthesis steps of FIG. 1; therefore, this bead only includes the inner Dexal polymer layer with streptavidin associated therewith. The bead in the left panel (FIG. 3A) was produced by a different scheme in which streptavidin is directly conjugated to the bead, and then reacted with TPA and Dexal to form a Dexal polymer coating layer about the streptavidin. In embodiments, each layer as described herein extends continuously around the substrate (such as polystyrene bead (20) of FIG. 1).

In particular, the bead shown in the left panel (FIG. 3A) was produced by two different synthesis schemes. First, the substrate such as 0072 bead was produced by combining a bead-COOH with EDAC, then adding streptavidin to the resultant mixture, and then adding TPA and Dexal to the resultant mixture to provide the bead shown. Second, the 19A bead (which is made of Marburg beads) was produced by combining a bead-CHO and streptavidin, and then adding TPA and Dexal to the resultant mixture to provide the bead shown.

When the beads shown in FIG. 3A were tested, both the 0072 and 19A beads showed a smaller curve at effective concentration. In addition, bead 0072 needed a higher bead concentration (10-20 mg/ml) due to lower streptavidin loading (Bead: SA ratio of 15:1).

The bead shown in FIG. 3B was specifically produced by combining a bead-COOH with hydrazine, then adding Dexal to the resultant mixture, and then adding streptavidin to the resultant mixture. This bead had a significant impact on curve size and was found to bind both free biotin and biotinylated antibody indiscriminately.

The embodiment shown in FIG. 3C was produced by combining bead-COOH with hydrazine, then adding Dexal to the resultant mixture, then adding streptavidin to the resultant mixture, then adding TPA and Dexal to the resultant mixture. This bead had a minimal impact on curve size and had good selectivity for free biotin binding, as discussed in greater detail herein below. In addition, this bead showed good biotin interference mitigation at 2-5 mg/ml beads.

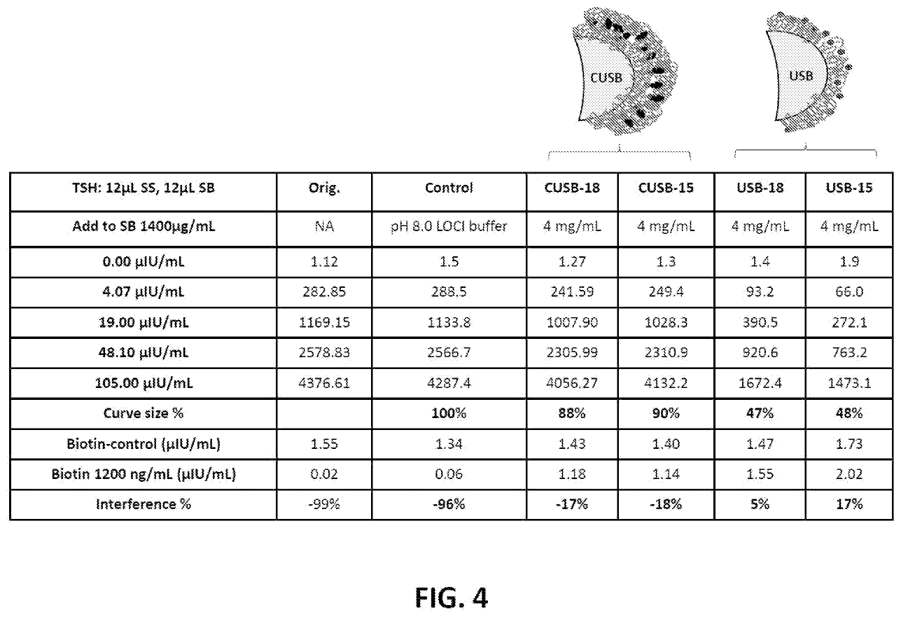

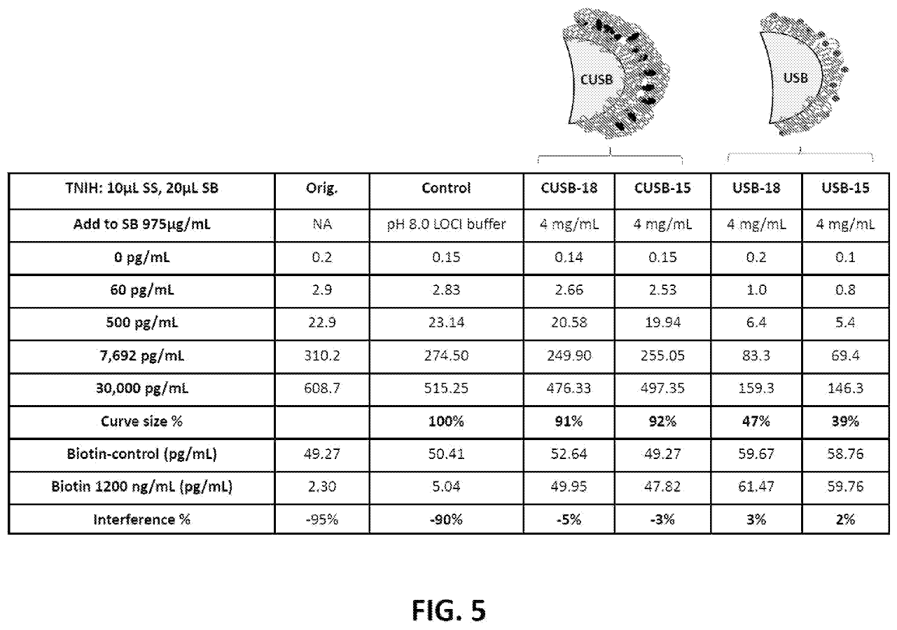

Specific data obtained with the biotin-trap beads is shown in FIGS. 4-10. The trap beads were added to the assay reagent(s) to scavenge free biotin molecules from patient samples to mitigate free biotin interference with biotinylated antibody or biotinylated protein binding to streptavidin in the reaction mixture. Streptavidin in the assay reagent is often immobilized on a solid support such as a surface or micro or nano beads (e.g., latex or magnetic particles). The LOCI® assay format was studied for the effectiveness of the trap beads, where they were added to the sensibead reagent at closer to the end of the assay. The streptavidin coated sensibeads bound both free and conjugated biotin, but the trap beads in excess amount preferentially absorbed free biotin, which led to biotin interference mitigation for LOCI® TSH (Thyroid-Stimulating Hormone) and TNIH (High Sensitivity Troponin I) assays (FIG. 4) and for FT4 (free thyroxine) and FT3 (free triiodothyronine) assays (FIG. 5).

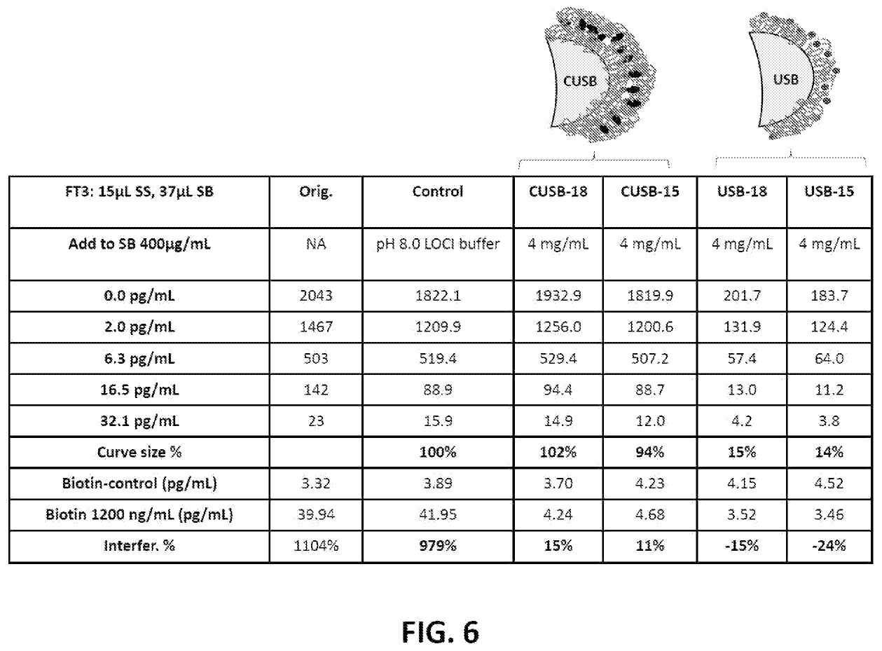

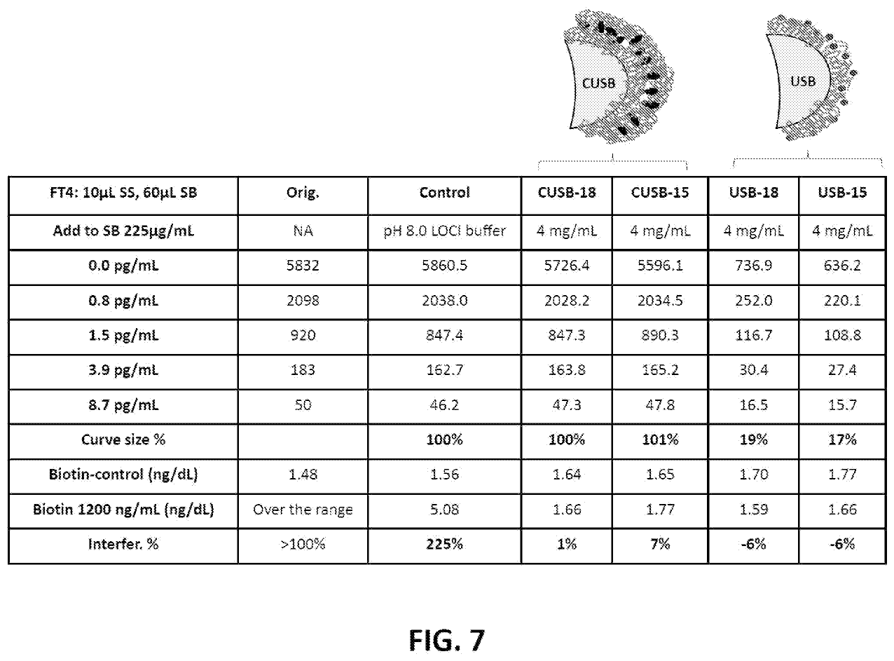

FIGS. 4-5 show the results for sandwich assays represented by TSH and troponin, respectively, and FIGS. 6-7 show the results of competitive assays represented by FT3 and FT4, respectively. These four figures (FIGS. 4-7) demonstrate that different designs of trap beads had different binding selectivities for free biotin versus protein-conjugated biotin. The term “CUSB” refers to covered sensibeads, and the term “USB” refers to un-covered sensibeads which don't have the top layer of Dexal to cover the streptavidin. Because the uncovered beads lack the top layer of Dexal, USB showed poor binding selectivity (i.e., these beads bind both free and conjugated biotin well), which resulted in less biotin interference mitigation and bigger curve size reduction.

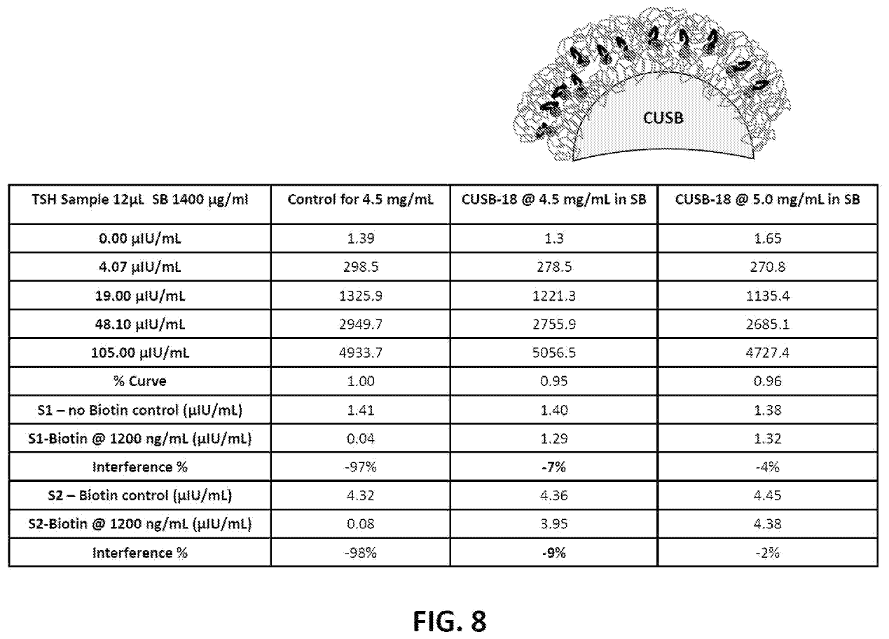

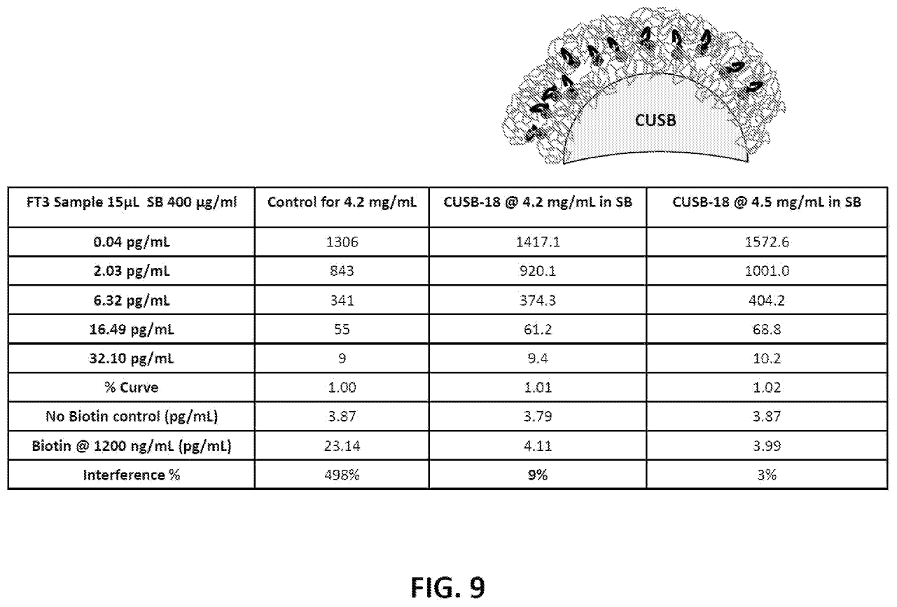

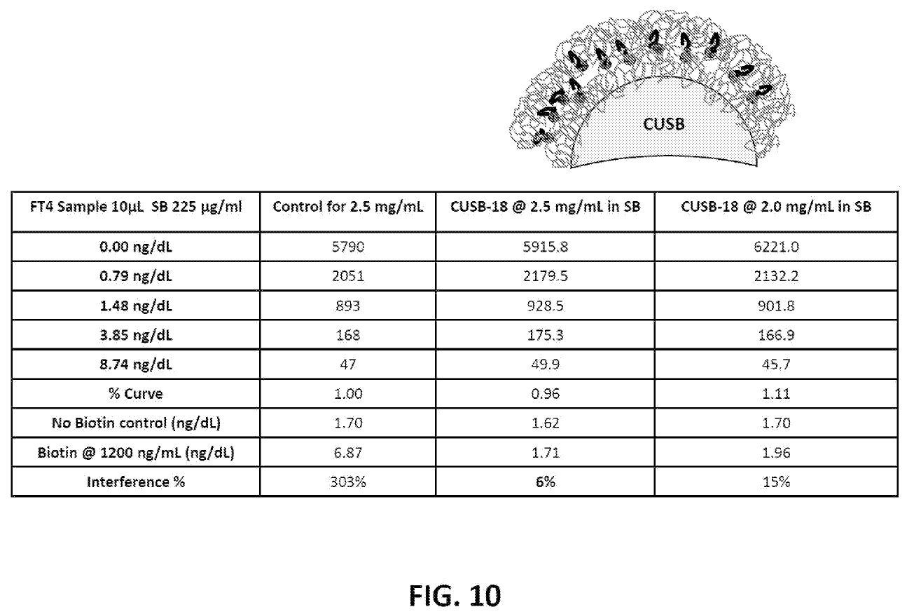

FIGS. 8-10 show the concentration optimization of CUSB spiked into the sensibead reagent for each assay. For the TSH assay (FIG. 8), the addition of 4.5 mg/mL CUSB was sufficient to lower the biotin interference from −97% to −9%. For the FT3 assay (FIG. 9), the addition of 4.2 mg/ml CUSB reduced the biotin interference from 498% to 9%. For the FT4 assay (FIG. 10), the addition of 2.5 mg/ml CUSB reduced the biotin interference from 303% to 6%. Meanwhile, the curve sizes remained similar to those without the CUSB spike.

Thus, in accordance with the present disclosure, there have been provided compositions, kits, and devices, as well as methods of producing and using same, which fully satisfy the objectives and advantages set forth hereinabove. Although the present disclosure has been described in conjunction with the specific drawings, experimentation, results, and language set forth hereinabove, it is evident that many alternatives, modifications, and variations will be apparent to those skilled in the art. Accordingly, it is intended to embrace all such alternatives, modifications, and variations that fall within the spirit and broad scope of the present disclosure.

1. A biotin-trap composition, comprising:
a particle having an outer surface;
an inner dextran aldehyde polymer coating layer disposed on at least a portion of the outer surface of the particle;
a biotin-specific binding partner conjugated to the inner dextran aldehyde polymer coating layer, wherein the biotin-specific binding partner is avidin or an analog thereof; and
at least one outer dextran aldehyde polymer coating layer disposed about the biotin-specific binding partner.

2. The biotin-trap composition of claim 1, further comprising tetraethylene pentaamine (TPA) or poly(ethylene amine) n (where n is 1 to 20) that binds to the biotin-specific binding partner and retains the at least one outer dextran aldehyde polymer coating layer disposed about the biotin-specific binding partner.

3. The biotin-trap composition of claim 1, wherein the particle is selected from the group consisting of a polystyrene bead, a latex bead, a magnetic particle, or a non-magnetic particle.

4. The biotin-trap composition of claim 1, wherein the outer surface of the particle has functional groups thereon for associating with the dextran aldehyde polymer.

5. The biotin-trap composition of claim 1, wherein the particle comprises a polystyrene bead having carboxylate functional groups on the outer surface thereof.

6. The biotin-trap composition of claim 1, wherein the biotin-specific binding partner is streptavidin.

7. A kit, comprising:
the biotin-trap composition of claim 1; and
at least one assay reagent for detecting the presence and/or concentration of a target analyte in a biological sample.

8. The kit of claim 7, wherein the at least one assay reagent is for use in a chemiluminescent detection system.

9. The kit of claim 7, wherein the at least one assay reagent comprises biotin.

10. The kit of claim 9, wherein the at least one assay reagent is a biotinylated target analyte-specific binding partner.

11. The kit of claim 10, wherein the biotinylated target analyte-specific binding partner is a biotinylated antibody.

12. A microfluidics device for determining the concentration of at least one target analyte in a sample, the microfluidics device comprising:
(i) an inlet channel through which a sample is applied; and
(ii) at least one compartment capable of being in fluidic communication with the inlet channel, wherein the at least one compartment contains:
(a) the biotin-trap composition of claim 1; and
(b) at least one assay reagent that comprises biotin.

13. The microfluidics device of claim 12, wherein the at least one assay reagent is for use in a chemiluminescent detection system.

14. The microfluidics device of claim 12, wherein the at least one assay reagent is a biotinylated target analyte-specific binding partner.

15. The microfluidics device of claim 14, wherein the biotinylated target analyte-specific binding partner is a biotinylated antibody.

16. The microfluidics device of claim 12, wherein the microfluidics device is configured for insertion in an automated diagnostic test instrument system.

17. A method of producing a biotin-trap composition, the method comprising the steps of:
(1) obtaining a particle having an outer surface with functional groups thereon;
(2) reacting the particle with dextran aldehyde polymer under conditions that form an inner dextran aldehyde polymer coating layer disposed on at least a portion of the outer surface of the particle;
(3) reacting the particle with a biotin-specific binding partner under conditions that conjugate the biotin-specific binding partner to the inner dextran aldehyde polymer coating layer, wherein the biotin-specific binding partner is avidin or an analog thereof;
(4) reacting the particle with tetraethylene pentaamine (TPA) or poly(ethylene amine) n (where n is 1 to 20); and
(5) reacting the particle with dextran aldehyde polymer under conditions that form an outer dextran aldehyde polymer coating layer.

18. The method of claim 17, wherein the particle is selected from the group consisting of a polystyrene bead, a latex bead, a magnetic particle, or a non-magnetic particle.

19. The method of claim 17, wherein the particle comprises a polystyrene bead having carboxylate functional groups on the outer surface thereof.

20. The method of claim 17, wherein the particle having the outer surface with functional groups thereon is formed by reacting a particle with carboxylate functional groups on the outer surface thereof with hydrazine.

21. The method of claim 17, wherein the biotin-specific binding partner is streptavidin.

22. A method of substantially removing free biotin from a biological sample, the method comprising the step of:
contacting the biological sample with the biotin-trap composition of claim 1; and
wherein the biological sample is contacted with the biotin-trap composition under conditions that allow free biotin present in the biological sample to substantially bind to the biotin-trap composition.

23. The method of claim 22, wherein the biological sample is selected from the group consisting of whole blood or any portion thereof, urine, saliva, sputum, cerebrospinal fluid, skin, intestinal fluid, intraperitoneal fluid, cystic fluid, sweat, interstitial fluid, extracellular fluid, tears, mucus, bladder wash, semen, fecal, pleural fluid, nasopharyngeal fluid, and combinations thereof.

24. A method of performing an assay for detecting the concentration of at least one target analyte in a biological sample, the method comprising the steps of:
(1) contacting, either simultaneously or wholly or partially sequentially:
the biological sample;
the biotin-trap composition of claim 1; and
at least one biotin-containing assay reagent;
wherein the biological sample is contacted with the biotin-trap composition under conditions whereby the biotin-trap composition specifically binds to free biotin present in the biological sample but does not substantially bind to the biotin-containing assay reagent;
(2) performing the assay using the at least one biotin-containing assay reagent; and
(3) determining the concentration of the at least one target analyte in the biological sample.

25. The method of claim 24, wherein the at least one biotin-containing assay reagent is a biotinylated target analyte-specific binding partner.

26. The method of claim 25, wherein the biotinylated target analyte-specific binding partner is a biotinylated antibody.

27. The method of claim 24, wherein the assay is a chemiluminescent detection assay.

28. The method of claim 24, wherein the biological sample is selected from the group consisting of whole blood or any portion thereof, urine, saliva, sputum, cerebrospinal fluid, skin, intestinal fluid, intraperitoneal fluid, cystic fluid, sweat, interstitial fluid, extracellular fluid, tears, mucus, bladder wash, semen, fecal, pleural fluid, nasopharyngeal fluid, and combinations thereof.
